(19) United States
(12) Patent Application Publication
(10) Pub. No.: US 2021/0386653 A1
(43) Pub. Date: Dec. 16, 2021

(54) COSMETIC TENSIONING COMPOSITION

(71) Applicant: Coty Inc., New york, NY (US)

(72) Inventors: Yuemei Zhang, Ramsey, NJ (US); Yan Wag, Parsippany, NJ (US)

(21) Appl. No.: 17/286,390

(22) PCT Filed: Oct. 24, 2018
(86) PCT No.: PCT/US2019/057579

§ 371 Date: Apr. 16, 2021

Related U.S. Application Data
(60) Provisional application No. 62/749,813, filed on Oct. 24, 2018.

(51) Int. Cl. A61K 8/87; A61K 8/25; A61K 8/73; A61K 8/365; A61K 8/34; A61Q 19/08

(52) U.S. Cl. CPC A61K 8/87; A61K 8/25; A61K 8/73; A61K 2800/524; A61K 8/34; A61K 8/345; A61Q 19/08; A61K 8/365

(57) ABSTRACT

The present application relates to cosmetic compositions and in particular, to skintightening compositions and to compositions that improve the appearance of the skin by providing skin-tightening improvements to the skin, for treatment of, for example, eye bags, facial wrinkles and other skin imperfections, the cosmetic compositions comprising a polyvalent silicate; at least one film former, and a carboxylic acid wherein the pH of the composition is in the range 6.0 to 11.2 wherein the composition provides a tightening effect on application to the skin. The present invention provides a composition which provides an efficient tightening product yet with good natural skin shine but without the white residue appearance associated with known products.

### FIELD

The present application relates to cosmetic tensioning compositions and, in particular, to skin-tightening compositions and to compositions that improve the appearance of the skin.

### BACKGROUND OF THE INVENTION

One of the most obvious signs of aging is the formation of visible wrinkles and an increase in puffiness of the area underneath the eyes. Consequently, a wide variety of products have been developed to hide or reduce the visible effect of these effects. Such products include topical cosmetics and paints to obscure the wrinkles and disguise the puffiness as well as more elaborate procedures involving the injection of compounds such as BOTOX™ under the skin in order to tighten the skin and prevent puffiness and wrinkles from occurring. Other procedures involve using topical treatments to hydrate and moisturise the skin, reducing the number of visible wrinkles by making the skin more supple and pliant. Other products contain active ingredients such as niacinamide designed to repair and rejuvenate the skin over time. Each of these products and procedures have associated disadvantages.

The application of BOTOX™ is an elaborate procedure requiring repeated visits to a specialist, and also tends to carry higher associated costs and risks than simpler topical treatments. The use of a cosmetic foundation layer and skin toners to conceal or reduce the appearance of fine lines, wrinkles, puffiness and other blemishes can have a short-term effect but once the makeup layer is removed, the skin underneath is the same as before the application of the makeup. Use of moisturizers and hydrating agents can have benefits and increase the health of the skin over time but these improvements take a relative long time and the benefits provided are incremental.

Consequently, attempts have been made to develop compositions which are aimed at reducing the appearance of fine lines, wrinkles and puffiness. One such product relies on the application of an adhesive film to the surface of the skin. The application of adhesive films to the skin results in the wrinkles being pulled upwards by the film, reducing the number of wrinkles. However, problems associated with existing adhesive film formers include irritation and discomfort for the user, caused by the contraction of the skin. Furthermore, the high visibility of the film which is often white in colour and distinguishable from the base skin tone of the face is a significant disadvantage as well as compatibility problems with other cosmetic products and the cracking of the film as it dries out over the course of several hours. Attempts to solve some of these problems have tended to exacerbate the others.

Sodium Silicate is commonly used as a film former in adhesive films. However, the films formed by sodium silicate tend to be very stiff and brittle. The addition of plasticizers may be an effective way to combat this problem, however the addition of such compounds results in less tightening of the film. The addition of covalent silicate promotes the tightening, but it tends to induce an undesirable white ashy look on skin. In addition to basic film forming compounds, recent developments include products that incorporate other additives such as UV absorbers, coloured pigments and skin lightening agents, for example.

### SUMMARY OF THE INVENTION

There remains a need for improved cosmetic compositions for producing skin tightening effects with low visible impact and lower pH that is more compatible with skin.

Thus, one of the main technical challenges in this field is to design an efficient skin-tightening composition that does not lead to the white appearance that arises on the skin and has lower pH than prior art.

The present invention seeks to alleviate the disadvantages of known skin tightening compositions by providing a composition that can tolerate wide pH range, does not result in the white appearance on the skin while still maintaining good skin tightening behaviour.

The present invention provides a composition as claimed in the appended claims.

Accordingly, the present invention provides a cosmetic composition comprising sodium silicate, pyrogenic silica, at least one film former, and a pH adjusting agent wherein the pH of the composition is in the range about 6.0 to about 11.2 wherein the composition provides a skin tightening effect after application to the skin. The composition of the present invention forms a film and provides a skin tightening effect when formed on the skin.

In some embodiments, the pH of the composition is in the range between about 6.0 to about 10, such as in the range 6.5 to 8.5. This has the significant advantage that the pH of the composition of the present invention is close to neutral and close to the pH of the skin to which it is to be applied. This means that the composition is skin friendly.

In some embodiments, the pH adjusting agent comprises a carboxylic acid, such as those selected from the group comprising citric acid and lactic acid.

The composition of the present invention has the advantage of providing a skin tightening effect after application to the skin, substantially without a white or ashy appearance resulting on the skin. Such white or ashy appearance is an unwanted feature associated with skin tightening products of the prior art.

Thus, it has surprisingly been found that a composition comprising pyrogenic silica (also known as fumed silica), alkaline silicate such as sodium silicate, a film former (plasticiser), such as polyurethane-35, and a carboxylic acid, such as citric acid, provide a significant skin tightening effect without the white appearance that arises on the skin associated with skin tightening products known in the art. In one embodiment of the present invention, the use of a carboxylic acid, such as citric acid or lactic acid, allows for the use of lower quantities of fumed silica, and also serves to lower the pH.

The present invention provides a composition which provides an efficient tightening product and does not lead to a white appearance on the skin. It also alleviates significant puffiness found in the area under the eyes.

Accordingly, the present invention provides cosmetic tensioning compositions as recited in the appended claims. Several aspects are recited in the dependent claims.

The compositions of the present invention do not include polyvalent silicate.

The compositions of the present invention have the significant advantage of efficiently tightening loose skin, decreasing wrinkles and puffiness under the eyes, thereby achieving smoother skin associated with a younger person.

It has been discovered by the inventor of the present invention that different areas of the face react differently to the same formulation. This is, on one hand, due to the fact that different areas of the face experience different levels of skin moisturization and, on the other hand, due to the fact that many of the facial expressions that humans make on a day-to-day basis result in the stretching/contracting of different areas of the skin.

Thus, some of the reasons for the whitening effect can be attributed to the movement of facial muscles over the course of the day that results in the dried film cracking/becoming more visible. The present invention provides a composition that produces a remarkable skin tightening effect that is also demonstrated to be long lasting.

The inventor has discovered that the compositions of the present invention are particularly useful when applied to the area under the eyes. It not only shows effective anti-wrinkle activity but also reduces puffiness in the undereye area of the facial skin. The composition of the present invention provides a combination of effects that, up until this point in time, have not been disclosed in any prior art. This advantage is in addition to the technical effect that due to the higher flexibility of the present invention as compared with the compositions of the prior art, the whitening effect of film drying is much reduced, especially for the under-eye area.

The under-eye area is of particular interest because it is highly moisturized and because it is usually compressed, not stretched, which improves film durability. Stretching of the film is related to higher and more rapid development of whiteness.

Examples of test results using the composition of the present invention are provided in the accompanying Figures. The clinical tests were carried out using a mixture of the novel invention in the presence/absence of skin toner (foundation). This is another aspect to the invention that is especially advantageous with regard to the state of the art, as most of the known products are typically, incompatible with other cosmetic products. The present invention however, while it does lose some of its efficiency with respect to skin-tightening and puffiness reduction, can be used on top of foundation and toner/concealer.

The testing scheme used was based on a visual grading scale with increasing severity of wrinkles/puffiness going from 1 (mild) to 10 (severe). The right and left under eye areas were evaluated independently for puffiness and wrinkles (before and after application of product).

The clinical grader assessed the visual puffiness of each under eye area independently using the 10-point scale shown below.

| NONE | MILD | | | MODERATE | | | SEVERE | | |
|---|---|---|---|---|---|---|---|---|---|
| 0 | 1 | 2 | 3 | 4 | 5 | 6 | 7 | 8 | 9 |

The clinical grader assessed the visual wrinkles of each under eye area independently using the 10-point scale shown below.

| NONE | MILD | | | MODERATE | | | SEVERE | | |
|---|---|---|---|---|---|---|---|---|---|
| 0 | 1 | 2 | 3 | 4 | 5 | 6 | 7 | 8 | 9 |

When used with foundation, the present invention showed a reduction from 6 to 4, with the effect diminishing in effectiveness over the course of 8 hours. When the formulation was applied on its own a reduction from 6 to 1 was observed, again with the effect lessening over several hours. The sample size of the study was 18 people.

Sodium Silicate is a film former on its own, but in order for it to be an effective cosmetic product, several types of additives need to be included in the formulation in order to keep the skin moisturized and comfortable and keep the formula stable. These additives, however, reduce the effectiveness of the skin-tightening properties of the sodium silicate. In the prior art, polyvalent silica has been used in order to restore some extent of the skin tightening effect. Surprisingly, the inventor has discovered that the addition of relatively small amounts of pyrogenic silica to sodium silicate (ratio 8:1 sodium silicate:pyrogenic silica) (range of weight ratio of sodium silicate:pyrogenic silicate is 40:1 and 1:1) has achieved remarkable effects of skin tightening and reducing puffiness in particular, in the under-eye area.

The composition also comprises carboxylic acid, such as citric acid or lactic acid. It has been found that citric acid is effective in reducing the pH of the composition which is otherwise at around pH 11 resulting from the alkaline sodium silicate and pyrogenic silica. This resulted in the formulation becoming thicker and viscous, and with a separate phase that looked like white particles, a technical problem which was solved with the addition of additives including Xanthan gum, Glycerine, Butylene Glycol, and polyurethane-35 first before adding the citric acid when forming the composition. Accordingly, the present invention also provides a method for forming the composition referred to above, the method steps being set out in the appended claims including the steps of:
(a) mixing sodium silicate and pyrogenic silica in predetermined amounts;
(b) adding polyurethane-35 to the mixture; and
(c) after step (b), then adding citric acid in pre-determined amounts. With the specific ingredients combination and addition sequence, a uniform and stable composition is formed having a pH in the range 6.5 to 9. The pH is lowered by addition of more citric acid to the composition.

### Definitions

Throughout this specification, the following terms are used and will be understood to have the following meaning:

Sodium Silicate—is a generic name for chemical compounds with the formula Na₂ₓSiO₂₊ₓ or (Na₂O)ₓ.SiO₂, such as sodium metasilicate Na₂SiO₃, sodium orthosilicate Na₄SiO₄, and sodium pyrosilicate Na₆Si₂O₇. The anions are often polymeric. These compounds are generally colorless transparent solids or white powders, and soluble in water in various amounts. Sodium silicate is also the technical and common name for a mixture of such compounds, chiefly the metasilicate, also called water glass, or liquid glass. In industry, the various grades of sodium silicate are characterized by their SiO₂:Na₂O weight ratio (which can be converted to molar ratio by multiplication with 1.032). The ratio can vary between 2:1 and 3.75:1. Grades with ratio below 2.85:1 are termed alkaline. The one used in the compositions of the present invention has the weight ratio 3.22, which is alkaline.

Pyrogenic/Fumed Silica;

CAS number 112945-52-5, produced in a flame, consists of microscopic droplets of amorphous silica fused into branched, chainlike, three-dimensional secondary particles which then agglomerate into tertiary particles. The resulting powder has an extremely low bulk density and high surface area.

### BRIEF DESCRIPTION OF THE DRAWINGS

The present application will now be described with reference to the accompanying drawings in which:

FIG. 1a shows Clinical Visual Grading (Puffiness) results for Intra-Subject Change in Moderate Group for i) Serum only and ii) Serum & Color Product;

FIG. 1b shows Clinical Visual Grading (Puffiness) results for Intra-Subject Change in Severe Group for i) Serum only and ii) Serum & Color Product;

FIG. 2a shows Clinical Visual Grading (Wrinkles) results for Intra-Subject Change in Moderate Group for i) Serum only and ii) Serum & Color Product; and

FIG. 2b shows Clinical Visual Grading (Wrinkles) results for Intra-Subject Change in Severe Group for i) Serum only and ii) Serum & Color Product.

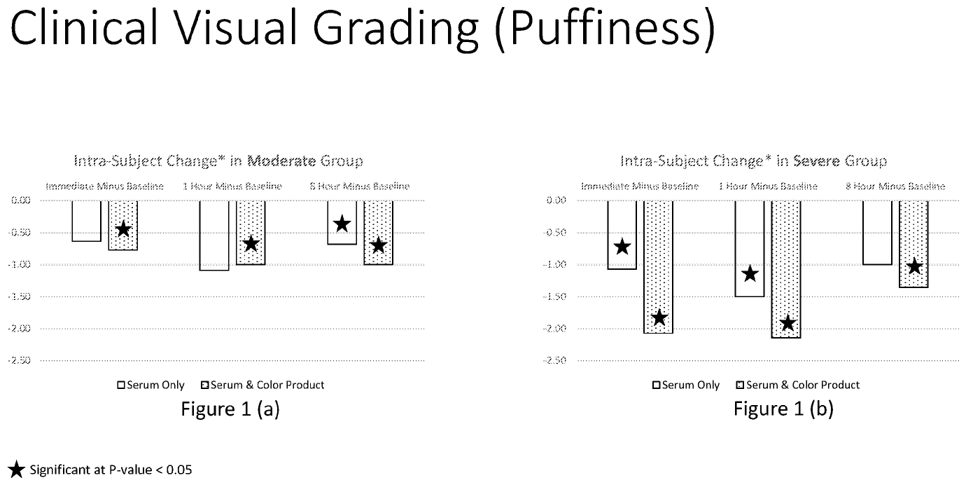

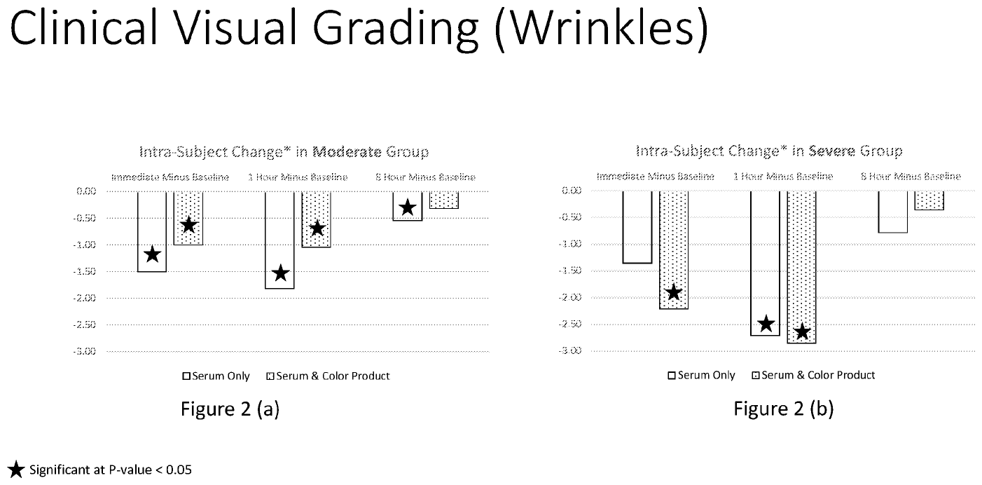

### DETAILED DESCRIPTION OF THE INVENTION

Historically, anti-wrinkle cosmetic products have relied heavily on silica and polyvalent silicate film formers in order to produce their asserted skin-tightening effect to reduce wrinkles and puffiness. These prior art compositions usually have a pH range of around 10-12, as the silica itself is strongly alkaline and it is taught in the prior art that lower pH leads to the formation of a more brittle and less flexible film on the skin, which in turn resulted in cracking, whitening and other obvious visual drawbacks. However, even at higher pH, many current market products still result in a whitening effect of the area of the skin onto which the composition is applied.

The present invention provides a skin tightening composition comprising sodium silicate and pyrogenic fumed silica with a solid weight ratio of pyrogenic silica:sodium silicate of about 1:5 based on the solids level since the solids level of sodium silicate is in the range of between 34-42%, preferably, 35-40% and most preferably, 36-39% and most preferably, 38-39% The technical advantage of the composition of the present invention is that an enhanced skin-tightening effect is achieved which has been found to smooth facial wrinkles over a period of up to 8 hours while simultaneously reducing puffiness. A combination of pyrogenic fumed silica and sodium silicate has been found to be surprisingly effective. While the mechanism of combination is not known, it is thought that the skin tightening effect may arise due to the fact that unlike previous compositions disclosed by prior art the current composition is not chemically cross-linked but instead physically cross-linked. This results in a more pliant and flexible film that allows the facial muscles underneath the skin to move without resulting in film cracking and whitening. The synergistic effect is shown in the Examples shown in Table 1 below. In the absence of pyrogenic fumed silica, the efficient skin-tightening effects is not achieved, whereas even small amounts of pyrogenic fumed silica will contribute to significantly increased skin tightening properties.

All amounts are in % w/w (weight of ingredient to weight of total composition).

TABLE 1

| Raw Material | Raw Material | | | | |
|---|---|---|---|---|---|
| Trade Name | INCI Name | CB8-5B1 | CB8-5C1 | CB8-5D1 | CB8-5E1 |
| DI water | DI water | 65.0 | 60.0 | 70.0 | 75.0 |
| O Sodium | sodium | 6.0 | 12.0 | 10.0 | 8.0 |
| Silicate | silicate, water | | | | |
| Baycusan C | Polyurethane | 12.0 | 12.0 | 8.0 | 5.0 |
| 1004/1 | 35, water | | | | |
| Glycerin | Glycerin | 1.5 | 3.5 | 1.6 | 3.3 |
| 99.7% usp | | | | | |
| 1,3-butylene | 1,3-butylene | 3.5 | 3.5 | 1.5 | 3.1 |
| glycol | glycol | | | | |
| Wacker HDK N20 | silica | 2.0 | 0.9 | 0.5 | 0.1 |
| Keltrol CG-T | Xanthan Gum | 0.8 | 0.8 | 0.8 | 0.3 |
| Citric Acid | Citric acid, | 9.0 | 6.5 | 7.0 | 5.0 |
| (20% water | water | | | | |
| solution) | | | | | |
| Phenoxyethanol | Phenoxyethanol | 0.2 | 0.8 | 0.6 | 0.2 |
| Total | | 100.0 | 100.0 | 100.0 | 100.0 |
| | Tightening feel | significant | moderate | moderate | slight |
| | Wrinkle | significant | moderate | moderate | slight |
| | smoothing | | | | |
| | effect | | | | |
| | Appearance | Moisturized | Moisturized | Moisturized | Moisturized |
| | | natural skin | natural skin | natural skin | natural skin |
| | pH | 7.2 | 7.47 | 7.66 | 8.37 |

pH of the composition is in the range 6.0 to 11.2

The ranges of the materials are indicated below with the total adding up to 100% w/w

TABLE 2

| Raw Material | Percentage % w/w |
|---|---|
| Deionised water | 60-75 |
| Sodium silicate | 6-12 |
| (38.6% water solution) | |
| Fumed Silica | 0.1-2 |
| Polyurethane Polymer | 5-12 |
| (41% emulsion) | |
| Xanthan Gum | 0.3-0.8 |
| Citric Acid (20% water | 5-9 |
| solution) | |
| 1,3-butylene glycol | 1.5-3.5 |
| Glycerine | 1.5-3.5 |
| 1,2-hexanediol and | 0-2 |
| Capryloyl glycol | |
| Phenoxy ethanol | 0.0-2.0 |
| Total | 100.00 |

Examples in Table 1 and 2 are tested on the back of a hand at the dosage of 2 mg/cm using finger to apply. 32 mg sample was applied in the 4*4 cm² area and allowed to dry for 3 minutes while the hand is laid flat, the stretch test was carried out by making a first which stretches the film that has formed on the skin from the application of the composition of the present invention. The result was observed as a visual test, without any instrument measurement.

With reference to Table 1, it can be seen that the composition tightening feel and wrinkle smoothing effect increase with the amount of pyrogenic silica.

It can be seen from the above Table 1 that providing a composition comprising sodium silicate, pyrogenic silica, citric acid, and polyurethane plays a vital role in the efficacy of the resulting composition. If pyrogenic fumed silica and citric acid are not used, it has been found that the tightening effect is reduced. The skin tightening effect provided by the composition of the present invention comprising pyrogenic silica, sodium silicate, citric acid and polyurethane is of commercial importance and valuable because it is far less visible on the skin than alternative market products when applied to the skin; this is likely due to the fact that polyurethane wet the surface of dry particles such as silica to prevent the white chalky look after the product is completely dried and in addition polyurethane also offers great adhesion on skin and improved flexibility to minimize cracking due to skin movement. The lack of or diminishment of the whitening effect that is known to occur when the prior art compositions are used on the skin, is a significant advantage of the present invention.

In addition to the skin-tightening benefits that the current invention achieves, the novel composition also has an additional technical effect of reducing the puffiness under the eyes, a common side effect of ageing along with wrinkle formation. This combination of effects is not disclosed in any of the prior art, and to our knowledge, there exists no single formulation that would alleviate both wrinkles and puffiness with a single topical treatment.

Additionally, the tolerance of skin tightening products for other cosmetic compositions has never been high, and indeed, as evidence of this, the use of other cosmetic products such as toner and foundation in conjunction with the compositions disclosed in prior art is discouraged. However, in the current composition, the use of cosmetics such as toner, foundation, while reducing the effect of the skin-tightening and puffiness reduction, does not prevent the current invention from fulfilling its described technical purpose.

Prior to the skin tightening composition of the present invention, most of the prior art skin-tightening products have had pH ranges of 10-12, which is close to being classified as ‘corrosive’ for the skin. Generally, lower pH values are not recommended as the use of sodium silicate in acidic media resulted in higher viscosity and gelation of the formulation. In this present invention, one embodiment is disclosed in which citric acid is included in the composition as well as a self-emulsified polymer, such as Polyurethane-35 (commercially available such as under the trade name, BAYCUSAN C1004, a commercially available product from Covestro AG (formerly Bayer Material Science AG), Leverkusen). This polyurethane product comprises 2-20% by weight of the composition of the present invention, such as in the range of 5˜12% by weight of the composition.

In another aspect, the present invention also relates to a method of preparation of a skin tightening a composition wherein the method comprises the step of adding citric acid in the presence of thickener and self-emulsified polyurethane-35 to maintain the stability of the formula. The citric acid reduced the pH of the solution to the range of 6-10, such as in the range 6.5-8.5. This improvement over the prior art results in a less alkaline composition that is more compatible with skin and less aggressive as compared with known cosmetic skin care products.

In some embodiments, silica is present in an amount within the range 0.1-5% w/w of the composition, 0.6%-32% of the solid contents, such as 0.2-2% w/w of the composition and 1.3-13% of the solids contents.

For citric acid, in some embodiments, 20% citric acid solution is present in an amount within the range of 1-10% of the composition and present in an amount with the range of 1.3-13% of the solids contents, such as present in a range of 5-9% and present in an range of 6.5-11.7% of the solid contents.

In some embodiments, the sodium silicate solution comprises Na₂O and SiO₂ with a 3.22 weight ratio of SiO₂:Na₂O suspended in a 34-42% water solution. For example, the sodium silicate solution comprises 4-20% of the total weight of the composition, such as 6-12%.

In addition to the pyrogenic fumed silica and sodium silicate, other additives may be included in the formulation of the present invention to form an effective skin tightening product.

The composition may also contain 0-2% xantham Gum, such as Keltrol CG-T, to provide a transparent composition. Xantham gum allows for the stabilization of the resultant film by significantly increasing the viscosity of the composition, preventing it from running off the area of skin to which it was applied.

In some embodiments, the composition also comprises 0.5-6% 0.5-20% 1,3-butylene glycol that similarly to Xanthan Gum acts as an anti-freeze agent to stabilize the formation of the film.

The composition may also comprise 0.5-6% glycerine, which is used in skincare to promote the smoothness and moisture content of the skin. This hydration can also help chronically reduce wrinkle visibility by making the skin more pliant.

Preservatives and stabilisers make up 0-2% of the total weight of the formulation and is used as a preservative and a stabilizer. Preservatives such as phenoxyethanol, Chlorophenesin, parabens can be used in the formulation.

An additional additive may comprise a film former, such as a high molecular weight polyurethane. Preferred polyurethane is Polyurethane-35 such as commercially available under the trade name BAYCUSAN C1004/1, a commercial product from Covestro AG (formerly, Bayer Material Science AG), Leverkusen. The polyurethane film former comprises 2-20% by weight of the formulation, such as in the range of 5-12%.

Water based PU usually demonstrates good affinity to dry pigment/particles, therefore water-based PU helps to wet/disperse the dry particle to minimize the matt/dry particle look. However, if a lot of PU is used, it can promote a glossy look which is undesirable. For this application, the ratio of PU used must be controlled since too much could result in interference with the sodium silicate polymer structure to weaken the skin tightening effect. Of course, it is to be understood that other polymers could wet dry pigment or particles as well such as polyester, etc. However, for the composition of the present invention, PU can be the plasticiser because of its great adhesion on the skin and elastic shape recovery capability, which are also beneficial properties to help with durability of the skin tightening effect.

The remaining weight percent is made up by deionised (DI) water.

It will be understood by those skilled in the art that some of the above additives are not necessary to achieve the desired technical skin-tightening effect. Compounds that promote skin health and/or that exhibit antimicrobial activity are not necessary for skin-tightening but do act in concert with the skin-tightening compounds to improve the overall appearance and health of the skin.

The resulting composition should then be applied to the skin in a regular and homogenous layer according to conventional manner. In some embodiments, the composition can be applied to a layer of clean, dry skin that is free of excessive oils. It should be applied in a single layer and allowed to dry.

### EXAMPLES

### Method of Preparation of the Skin Tightening Composition of the Present Invention

### Example 1

The preparation method for the present invention will now be described with reference to Table 3 below.

Initially, deionised (DI) Water, O Sodium Silicate and Fumed Silica are combined in the amounts and ratios indicated in Table 3 below and the ingredients were mixed at a medium speed, such as about 800 to about 1500 rpm, for 5-10 minutes until a uniform/homogenous mixture is obtained. An overhead mixer and propeller blade were used, in this particular Example, IKA® RW 20 with prop/propeller blade was used for stirring until homogeneous. Of course, this is only one method and apparatus that can be used.

This mixing stage followed by the addition of polyurethane, continuously mixing the composition at a medium speed, such as about 800 to about 1500 rpm, until homogenous.

Simultaneously, pre-wet xanthan gum, Glycerine and 1,3-butylene glycol are mixed in a separate container before being added to the main composition. This leads to a significant increase in the viscosity of the composition. The composition is mixed at a higher rate until no further increases in viscosity occur.

Finally, 20% citric acid in water as well as a preservative such as phenoxy ethanol are added at high speed, such as about 1000 rpm to about 2000 rpm according to the viscosity changes to ensure that the composition is emulsified uniformly.

The final composition is then mixed at high speed, such as about 1500 to about 3000 rpm speeds, for 10-20 minutes to ensure a homogenous emulsion. The emulsion is an oil in water emulsion where the internal oil phase is the polyurethane in an external aqueous phase.

It was found that when citric acid is added into the system with water fumed silica, sodium silicate and Polyurethane-35 (Baycusan C1004/1) the system would gel up and insoluble small, sand-sized particles appear. The particles cannot be broken down in the later steps. This is not only an indication of non-uniform/unstable system, it is also very hard to break the sandy particles during application process and the product loses some visual appeal. For formula 87B3P, the Baycusan C1004/1 was not added until after the citric acid, and a similar phenomenon is observed. This demonstrates that the sandy particles are not because of the Baycusan C1004, which is polyurethane. Therefore, the inventor believes it is because of the reaction between the alkaline Sodium silicate with citric acid.

The following is the formula used in Example 1 with the process steps set out to allow the final composition of the finished system to have a uniform/stable phase.

TABLE 3

| | | | |
|---|---|---|---|
| Order of | Raw Material | Raw Material | |
| Addition | Trade Name | INCI Name | 97B2-suC |

Phase A: Weigh and add A1, A2 and A3 in sequence into the container
while mixing

| | | | |
|---|---|---|---|
| A1 | DI water | DI water | 67.7 |
| A2 | O Sodium | sodium | 8 |
| | Silicate | silicate | |
| A3 | (Pyrogenic | Fumed silica | 1.2 |
| | Silica - Fumed | | |
| | Silica) Wacker | | |
| | HDK N20 | | |

Mix at medium speed, such as 800 to 1500 rpm, for 5~10 minutes
until uniform
Phase B: Weigh and add B into the system while mixing,

| | | | |
|---|---|---|---|
| B1 | Baycusan | Polyurethane | 10 |
| | C 1004 | 35 | |

Mix at medium speed, such as 800 to 1500 rpm, for 5~10 minutes
until uniform
Weigh and premix Cs into a separate container to pre-wet Xanthan
Gum into the liquid
Add C mixture into the system while mixing, system viscosity
will increase, increase speed accordingly

| | | | |
|---|---|---|---|
| C1 | Keltrol CG-T | Xanthan Gum | 0.5 |
| C2 | Glycerin 99.7% | Glycerin | 2.4 |
| | usp | | |
| C3 | 1,3-butylene | 1,3-butylene | 2.3 |
| | glycol | glycol | |

Mix at (medium speed), such as about 1000 to about 1500 rpm depending
on viscosity increase for 10 minutes or until xanthan gum is completely
activated, and the viscosity does not have any further drastic
increase.
Add Ds at high speed, such about 1500 to about 2000 rpm.

| | | | |
|---|---|---|---|
| D1 | Phenoxy | Phenoxy | 0.9 |
| | ethanol | ethanol | |
| D2 | Citric Acid | Citric | 7 |
| | (20% water | Acid, water | |
| | solution) | | |

Mix at high speed, such as about 1500 to about 3000 rpm for 10-20
minutes; and the system looks like a uniform serum.

| | |
|---|---|
| Total | 100 |

The following description sets forth other experiments which were carried out in which phase separation occurred and these experiments are included here for comparison and for reference.

From formula 87B2P, it was found that when citric acid is added into the system with water fumed silica, sodium silicate and Baycusan C1004, system gels up and non-compatible sand sized particles appears, and the particle cannot be broken down in the later steps. This is not only an indication of non-uniform/unstable system, it is also very hard to break the sandy particles during application process and the product does not have an appealing appearance. For formula 87B3P, the Baycusan C1004/1 was not included in the composition before adding the citric acid, and a similar phenomenon of phase separation was observed. This demonstrates that the sandy particle is not because of the Baycusan C1004/polyurethane. Therefore, the inventor believes it is because of the reaction between the alkaline Sodium silicate with citric acid.

### Example 2

The preparation method for the present invention will now be described with reference to Table 4 below. The process steps as set out in Example 1 were followed as set out in Example 1 above.

Initially, deionised (DI) Water, O Sodium Silicate and Fumed Silica are combined in the amounts and ratios indicated in Table 4 below and the ingredients were mixed at medium speed, such as about 800 to about 1500 rpm, for 5-10 minutes until a uniform/homogenous mixture is obtained.

This mixing stage followed by the addition of polyurethane-35, continuously mixing the composition at medium speed, such as about 800 to about 1500 rpm, until homogenous.

Process: Raw materials were added in the order of the sequence as listed from top to bottom of the table. The composition was mixed for about 10 minutes between each addition.

TABLE 4

| Raw Material | Raw Material | | | | |
|---|---|---|---|---|---|
| Trade Name | INCI Name | 87B2p | Observed | 87B3p | Observed |
| DI water | DI water | 66.9 | | 67 | |
| (Pyrogenic Silica - | Fume silica | 1.2 | | 1.2 | |
| Fumed Silica) | | | | | |
| Wacker HDK N20 | | | | | |
| O Sodium Silicate | sodium silicate | 8.0 | | 8.0 | |
| Baycusan C 1004 | Baycusan C 1004 | 10.7 | | | |
| Citric Acid (20% | Citric Acid (20% | 7 | PH~8gel up, | 7 | PH~8 gel up, |
| water solution) | water solution) | | sandy particles | | sandy particles |
| | | | appears (phase | | appears (phase |
| | | | separation) | | separation, stop |
| | | | | | the batch here |
| Baycusan C 1004 | Polyurethane 35 | | | 10.7 | |
| Keltrol CG-T | Xanthan Gum | 0.5 | viscosity | | |
| | | | increase | | |
| Phenoxy ethanol | Phenoxy ethanol | 1 | viscosity | 1 | |
| | | | increase | | |
| Glycerine 99.7% usp | Glycerine | 2.4 | viscosity | 2.4 | |
| | | | decrease | | |
| 1,3-butylene glycol | 1,3-butylene glycol | 2.4 | viscosity | 2.4 | |
| | | | decrease | | |
| Keltrol CG-T | Xanthan Gum | | viscosity | 0.4 | |
| | | | increase | | |
| Citric Acid (20% | Citric Acid (20% | | | | |
| water solution) | water solution) | | | | |
| Total | | 100 | PH~8gel up | 100 | |
| | | | with sandy | | |
| | | | particles | | |

### pH Study

In order to study how pH affects the efficacy of the present invention, two variations of the described composition were neutralized to two different pH levels, 8.6 and 6 as shown in Table 5 below. Both formula retained the desired viscosity, consistency and tightening effect. The in vivo test shows that the formulation with a pH of 6 was more prone to drying out and cracking, which resulted in a more obvious whitening of the area of the skin onto which the composition had been applied. Since citric acid is a white powder, it is assumed that too much acidic additive will result in the composition drying out more rapidly, resulting in the above described whitening effect. Consequently, further tests were carried out using formulations having a pH of 8.6 although there is potential for compositions with lower pH to be investigated for potential benefits.

It is understood that the optimum pH is at a pH of between 6 and 8.6, such as at 6.5-8.5.

TABLE 5

| Raw Material | Raw Material | | |
|---|---|---|---|
| Trade Name | INCI Name | 93B | 94A |
| DI water | DI water | 67.0 | 66.1 |
| (Pyrogenic | Fume silica | 1.2 | 1.2 |
| Silica - Fumed | | | |
| Silica) Wacker | | | |
| HDK N20 | | | |
| O Sodium | sodium | 8.0 | 7.9 |
| Silicate | silicate | | |
| Keltrol CG-T | Xanthan Gum | 0.4 | 0.4 |
| Phenoxy | Phenoxy | 1.0 | 1.0 |
| ethanol | ethanol | | |
| Glycerin | Glycerin | 2.4 | 2.3 |
| 99.7% usp | | | |
| 1,3-butylene | 1,3-butylene | 2.4 | 2.3 |
| glycol | glycol | | |
| Baycusan C | Polyurethane | 10.7 | 10.6 |
| 1004/1 | 35 | | |
| Citric Acid | Citric Acid, | 7.0 | 8.2 |
| (20% water | water | | |
| solution) | | | |
| | | 100.0 | 100.0 |
| | pH | 8.6 | 6 |

### Examples 3 and 4

In some embodiments, fumed Silica is Wacker HDK N20; Polyurethane Polymer is Baycusan C 1004; xantham Gum is Keltrol CG-T and 1,2-hexanediol and capryloyl glycol are provided as Symdiol 68. Other acidic additives can also be included.

| Raw Material | Raw Material | | |
|---|---|---|---|
| Trade Name | INCI Name | 93B | 96A |
| DI water | DI water | 67.0 | 70.1 |
| (Pyrogenic | Fume silica | 1.2 | 1.2 |
| Silica - Fumed | | | |
| Silica) Wacker | | | |
| HDK N20 | | | |
| O Sodium | sodium | 8.0 | 8.4 |
| Silicate | silicate | | |
| Keltrol CG-T | Xanthan Gum | 0.4 | 0.4 |
| Phenoxy | Phenoxy | 1.0 | 1.0 |
| ethanol | ethanol | | |
| Glycerin | Glycerin | 2.4 | 2.5 |
| 99.7% usp | | | |
| 1,3-butylene | 1,3-butylene | 2.4 | 2.5 |
| glycol | glycol | | |
| Baycusan C | polyurethane | 10.7 | 11.2 |
| 1004/1 | 35 | | |
| Citric Acid | Citric Acid, | 7.0 | |
| (20% water | water | | |
| solution) | | | |
| Lactic acid | Lectic acid | | 2.7 |
| Total | | 100 | 100 |

A liquid acid, lactic acid was tested by adding to system 96A directly without dilution in water. Compared to sample 93B, sample 96A was more viscous, still tightens well, the composition is very stable with no water separation. Sample 93B showed some water separation for some batches and the inventor believes that this might due to the water added in the last step with citric acid solution and there was not enough sheer to bring the water into the system. When lactic acid is used without water at the last step, the resulting stable system confirms this assumption as to the reason for the water separation in some instances. Therefore, longer sheer time/mixing time is provided at the last step of preparing the standard formula comprising citric acid.

1. A cosmetic composition comprising sodium silicate, pyrogenic silica, at least one film former, and a pH adjusting agent wherein the pH of the composition is in the range 6.0 to 11.2 wherein the composition provides a skin tightening effect after application to the skin.

2. A composition as claimed in claim 1 wherein the pH of the composition is in the range between 6.0 to 10.

3. (canceled)

4. A cosmetic composition as claimed in claim 1 wherein the composition provides a tightening effect after application to the skin, substantially without a white or ashy appearance on the skin.

5. A composition as claimed in claim 1 wherein the pyrogenic silica is present in an amount within the range 0.1-5% w/w of the composition, 0.6%-32% of the solids contents.

6-9. (canceled)

10. A composition as claimed in claim 1 wherein the sodium silicate comprises Na₂O and SiO₂ with a 3.22 weight ratio of SiO₂:Na₂O suspended in an aqueous solution.

11. A composition as claimed in claim 1 wherein the sodium silicate solution is in the range of 34-42% solids, and preferably, comprises 4-20% w/w of the total weight of the composition.

12. A composition as claimed in claim 1 wherein the composition also comprises a plasticiser.

13. A composition as claimed in claim 12 wherein the plasticiser is selected from the group consisting of glycerine, propylene glycol, butylene glycol and propanediol, and mixtures thereof.

14. (canceled)

15. A composition as claimed in claim 1 wherein the composition also comprises 0-2% xantham Gum to provide a transparent composition to allow for the stabilization of the film by significantly increasing the viscosity of the composition, preventing it from running off the area of skin to which it was applied.

16. A composition as claimed in claim 1 wherein the composition comprises a preservative.

17. A composition as claimed in claim 16 wherein the preservative is phenoxy ethanol.

18. (canceled)

19. A composition as claimed in claim 1 wherein the composition comprises an antimicrobial agent.

20. A composition as claimed in claim 19 wherein the antimicrobial agent is present in an amount in the range 0-2% by weight of the composition.

21. A composition as claimed in claim 1 wherein the acid comprises citric acid, present in an amount of 5-9% w/w 20% citric acid aqueous solution.

22-24. (canceled)

25. A composition as claimed in claim 1 comprising the following ingredients in an amount in the range indicated: (all amounts are in % w/w weight of the ingredient as total weight of the composition):
Pyrogenic Silica 0.1-2%
Sodium Silicate (38.6% water solution) 4-20%
Citric Acid (20% water solution) 5-9%
Polyurethane (41% water emulsion) 2-20%; and optionally: any one or more of the following:
Xanthan 0-2%
Polyhydric alcohol plasticizers 0.5-20%, optionally, the polyhydric alcohol plasticizers comprising 1,3 butylene glycol in an amount in the range 0.5-6% and Glycerin in an amount in the range 0.5-6%,
Phenoxy Ethanol 0-2%; and
1,2 Hexanediol and Capryloyl glycol 0-2%.

26. A composition as claimed in claim 1 comprising the following ingredients in the amounts indicated (all amounts are in % w/w weight of the ingredient as total weight of the composition):

| Raw Material | Percentage % w/w |
|---|---|
| Sodium silicate | 6-12 |
| (34-42%% water solution) | |
| Fumed Silica | 0.1-2 |
| Polyurethane Polymer | 5-12 |
| Citric Acid (20% water | 5-9 |
| solution) | |

27. A composition as claimed in claim 25 comprising the following additional ingredient in the amounts indicated (all amounts are in % w/w weight of the ingredient as total weight of the composition):

| Raw Material | Percentage % w/w |
|---|---|
| Deionised water | 60-75 |

28. A composition as claimed in claim 26 comprising the following additional ingredient(s) in the amounts indicated (all amounts are in % w/w weight of the ingredient as total weight of the composition):

| Raw Material | Percentage % w/w |
|---|---|
| Xanthan Gum | 0.3-0.8 |
| 1,3-butylene glycol | 1.0-15% |
| Glycerine | 1.5-3.5 |
| 1,2-hexanediol and | 0-2 |
| Capryloyl glycol | |
| Phenoxy ethanol | 0.0-2.0 |

29. A cosmetic composition comprising sodium silicate, pyrogenic silica, at least one film former, and a pH adjusting agent wherein the pH of the composition is in the range 6.0 to 11.2 wherein after application to the skin, the composition provides a skin tightening effect, substantially without a white or ashy appearance on the skin.

30. A method of preparation of the composition of claim 1 comprising the following steps:
(a) mixing sodium silicate and pyrogenic silica in pre-determined amounts;
(b) adding a film former to the mixture; and
(c) after step (b), adding a pH adjusting agent to achieve a pH in the range of between 6.0 to 11.2

31-33. (canceled)
